(19) United States
(12) Patent Application Publication
(10) Pub. No.: US 2026/0234461 A1
(43) Pub. Date: Aug. 13, 2026

(54) COMPOSITE EMULSIFIER AND OIL-BASED DRILLING FLUID

(71) Applicants: China Petroleum & Chemical Corporation, Beijing (CN); Sinopec Oilfield Service Corporation, Beijing (CN); Sinopec Shengli Petroleum Engineering Co., Ltd, Dongying, Shandong (CN); Drilling Fluid Technology Service Center of Sinopec Shengli Petroleum Engineering Co., Ltd, Dongying, Shandong (CN)

(72) Inventors: Xuxin WAN, Dongying, Shandong (CN); Ke LI, Dongying, Shandong (CN); Haibin LI, Dongying, Shandong (CN); Wenbo LI, Dongying, Shandong (CN); Chengjun WANG, Dongying, Shandong (CN); Mingxin MA, Dongying, Shandong (CN); Wei WANG, Dongying, Shandong (CN); Chongchong WANG, Dongying, Shandong (CN); LEI YU, Dongying, Shandong (CN); Bo LI, Dongying, Shandong (CN)

(73) Assignees: China Petroleum & Chemical Corporation, Beijing (CN); Sinopec Oilfield Service Corporation, Beijing (CN); Sinopec Shengli Petroleum Engineering Co., Ltd, Dongying, Shandong (CN); Drilling Fluid Technology Service Center of Sinopec Shengli Petroleum Engineering Co., Ltd, Dongying, Shandong (CN)

(21) Appl. No.: 19/469,471

(22) PCT Filed: Apr. 26, 2024
(86) PCT No.: PCT/CN2024/090082

§ 371 Date: Sep. 26, 2025

(30) Foreign Application Priority Data
Apr. 27, 2023 (CN) 202310477151.5

(51) Int. Cl. C09K 8/26; C09K 8/05; C09K 23/16

(52) U.S. Cl. CPC C09K 8/26; C09K 8/05; C09K 23/16

(57) ABSTRACT

Described are a composite emulsifier and an oil-based drilling fluid. The composite emulsifier includes a main emulsifier and an auxiliary emulsifier. The auxiliary emulsifier is a sulfonated substance and/or carboxylation product of the main emulsifier. The composite emulsifier has a good emulsification effect and good electrical stability and thermal stability, and can greatly reduce difficulties in compounding at a drilling site.

### CROSS-REFERENCE TO RELATED APPLICATIONS

The application claims the benefit of the China patent application No. “202310477151.5”, filed on Apr. 27, 2023, the content of which is specifically and entirely incorporated herein by reference.

### TECHNICAL FIELD

The invention relates to the technical field of oil-based drilling fluid emulsifiers, in particular to a composite emulsifier and an oil-based drilling fluid.

### BACKGROUND ART

The oil-based drilling fluids have excellent properties such as strong suppression, anti-pollution, lubrication, salt resistance and low reservoir damage, and are used more widely in water-sensitive formations, shale formations, deep wells, giant thick salt paste layers, salt-stone strata and the like. The emulsifier is one of the most critical treatment agents for the oil-based drilling fluids, it mainly serves to ensure the emulsion stability of the water-in-oil drilling fluids, thereby improving the rheological property of the oil-based drilling fluids. Currently, the emulsifier composition of oil-based drilling fluids is mostly composed of a main emulsifier and an auxiliary emulsifier, which cooperate with the hydrophilic surfactants based on the different structures; however, when the surfactants with different structures form an interfacial film, the integration degree is poor due to the diverse structures, thus it is not beneficial to the strength maintenance and stabilization of the emulsion. In addition, the existence of a main emulsifier and an auxiliary emulsifier will also increase the difficulty of the on-site operations.

CN115785920A discloses an emulsifier for water-in-oil emulsion drilling fluid, which is prepared by carrying out cyclization reaction by selecting fatty acid and organic amine as the reaction monomer, it is ensured that the emulsifier can be kept stable under the high-temperature environment by taking advantage of the five-membered heterocyclic ring structure in the molecular structure of the emulsifier, but the demulsification voltage of the emulsifier is below 400V. CN105907382A discloses an emulsifier based on an oil-based drilling fluid, the emulsifier contains tall oil fatty acid, polyamine, chloroactic acid amide, surfactants and organic solvents, it is compounded by adding the surfactants, but the invention merely discloses the temperature resistance of the emulsifier after an aging process at 150° C.

Therefore, it is urgent to solve the technical problems concerning how to reduce the difficulty of field operation and improve the electrical stability and temperature resistance of the oil-water emulsion system in the oil-based drilling fluid.

### SUMMARY OF THE INVENTION

The invention aims to solve the problems in the prior art with respect to poor stability of an oil-water emulsification system and high field operation difficulty, and provides a composite emulsifier and an oil-based drilling fluid, the composite emulsifier has excellent emulsification property, desirable stability, and low preparation and operation difficulty.

In order to achieve the above object, the first aspect of the present invention provides a composite emulsifier comprising a main emulsifier and an auxiliary emulsifier, is characterized in that the auxiliary emulsifier is a sulfonated substance and/or carboxylation product of the main emulsifier.

The second aspect of the present invention provides an oil-based drilling fluid comprising a base oil, and a treatment agent comprising the composite emulsifier according to the first aspect.

The emulsifier component of an oil-based drilling fluid mainly consists of a main emulsifier and an auxiliary emulsifier, wherein the main emulsifier has a certain lipophilicity, and the auxiliary emulsifier exhibits a certain hydrophilicity, but the conventional main emulsifier and the auxiliary emulsifier are provided by the surfactants with different structures, so that the on-site compounding difficulty is high, and the emulsifying system has a poor stability. In the composite emulsifier provided by the invention, the auxiliary emulsifier is a sulfonated substance and/or a carboxylation product of the main emulsifier, it can be directly used in a drilling fluid, and greatly reduces the difficulty of compounding at a drilling site. In the invention, due to the synergistic action of the main emulsifier and the auxiliary emulsifier, the oil-water interfacial tension can be effectively reduced, and a stable oil-water emulsification system can be formed. The composite emulsifier is used in an oil-based drilling fluid, the demulsification voltage may reach 674V or more, and it ensures the low high-temperature high-pressure filtration loss, thus the composite emulsifier is applicable to the emulsification of the different oil-based drilling fluids such as gas oil, white oil and diesel. In addition, the demulsification voltage of the composite emulsifier provided by the invention after an aging process at 1800 and 200° C. can be kept above 518V and 432V respectively, thus the composite emulsifier has excellent electrical stability and thermal stability.

### BRIEF DESCRIPTION OF THE DRAWINGS

FIG. 1 illustrates an infrared absorption spectrogram of the main emulsifier prepared in Example 1 of the present invention;

FIG. 2 shows a mass spectrogram of the main emulsifier prepared in Example 1 of the present invention;

FIG. 3 illustrates an infrared absorption spectrogram of the composite emulsifier A1 prepared in Example 1 of the present invention;

FIG. 4 shows a mass spectrogram of the composite emulsifier A1 prepared in Example 1 of the present invention.

### DESCRIPTION OF THE PREFERRED EMBODIMENT

The terminals and any value of the ranges disclosed herein are not limited to the precise ranges or values, such ranges or values shall be comprehended as comprising the values adjacent to the ranges or values. As for numerical ranges, the endpoint values of the various ranges, the endpoint values and the individual point values of the various ranges, and the individual point values may be combined with one another to produce one or more new numerical ranges, which should be deemed to have been specifically disclosed herein.

The first aspect of the present invention provides a composite emulsifier comprising a main emulsifier and an auxiliary emulsifier, wherein the auxiliary emulsifier is a sulfonated substance and/or carboxylation product of the main emulsifier.

In the composite emulsifier provided by the invention, the auxiliary emulsifier is a sulfonated substance and/or carboxylation product of the main emulsifier, it can be directly used in a drilling fluid, and greatly reduces the difficulty of compounding at a drilling site. The main emulsifier and the auxiliary emulsifier in the composite emulsifier provided by the invention have good integration, can obviously enhance the emulsification effect, the demulsification voltage may reach 674V or more, the demulsification voltage of the composite emulsifier after an aging process at 1800 and 2000 can be kept above 518V and 432V respectively, and the composite emulsifier has excellent electrical stability and thermal stability. In the prior art, when the surfactants with different structures and hydrophilicities are combined as an emulsifier, because the different surfactants have diverse structures, it is difficult to integrate the surfactants, the emulsifier has poor stability, the emulsifier generally need to be prepared through the on-site operation, thereby increasing the difficulty of the on-site operation.

According to the invention, the demulsification voltage of the composite emulsifier in a standard oil-based emulsion under the condition of a dosage of 5 wt % is preferably within the range of 50-400V, more preferably within the range of 80-280V, further preferably within the range of 200-280V, wherein the standard oil-based emulsion is composed of 5 #white oil and CaCl₂) aqueous solution having a concentration of 25 wt %, the volume ratio of oil-water is 80:20; the dosage refers to the mass fraction of the addition amount of an emulsifier based on the total mass of oil and water. The inventors of the present invention have discovered in researches that the composite emulsifier has a suitable demulsification voltage in a standard oil-based emulsion, the demulsification voltage within the preferred range is favorable for application of an emulsifier in the oil-based drilling fluid in practice, and the emulsifier has excellent emulsification performance. The demulsification voltage of an emulsifier in standard oil-based emulsions is not the higher the better, because the drilling fluids in practice have a relatively complicated composition, when the demulsification voltage of an emulsifier in the standard oil-based emulsions is too high, the emulsifier exhibits an excessively high polarity in the actual oil-based drilling fluids, the formed oil droplets are too tiny, which may result in increased plastic viscosity and dynamic shear force, and poor electrical stability and thermal stability.

According to some preferred embodiments of the invention, the emulsification ratio of the composite emulsifier in the standard oil-based emulsion is larger than or equal to 90%, more preferably within the range of 95-100%.

According to the present invention, preferably, the molar ratio of the auxiliary emulsifier to the main emulsifier in the composite emulsifier is (0.25-1):1, more preferably, the molar ratio of the auxiliary emulsifier to the main emulsifier in the composite emulsifier is (0.4-0.8):1. When the molar ratio the auxiliary emulsifier to the main emulsifier falls into the preferred composition range, it is advantageous to further exert the synergistic effect of main emulsifier and auxiliary emulsifier, and improve the stability of the oil-water emulsification system.

In the present invention, “the auxiliary emulsifier is a sulfonated substance and/or carboxylation product of the main emulsifier” means that a sulfonic acid group and/or a carboxylic acid group is introduced into the main emulsifier through the sulfonation reaction or the carboxylation reaction, the specific reaction modes are well-known among those skilled in the art.

According to the present invention, a total molar amount of the sulfonic acid groups and/or carboxylic acid groups in 1 mol of the composite emulsifier is preferably within the range of 0.1-1.2 mol, more preferably within the range of 0.3-0.6 mol, for example, it may be 0.3 mol, 0.35 mol, 0.4 mol, 0.45 mol, 0.5 mol, 0.55 mol, 0.6 mol and the other typical and non-limiting molar amount or a random value within the range consisting of any two molar ratios. In the invention, the molar amount of sulfonic acid groups is calculated in terms of the group represented by

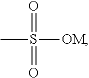

and the molar amount of carboxylic acid groups is calculated in terms of the group represented by

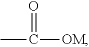

wherein M is an alkali metal or ammonium group introduced in the sulfonation reaction or the carboxylation reaction, it is well-known among those skilled in the art, for instance, M may be Na, K or ammonium.

In the invention, an organic element analyzer is used for testing the element composition in the main emulsifier and composite emulsifier, the content of sulfonic acid groups and/or carboxylic acid groups in the auxiliary emulsifier is calculated according to the change of molar amounts of S/C elements.

The structure of said main emulsifier is not particularly limited in the invention, it may be a conventional main emulsifier having lipophilicity in the field, preferably, the main emulsifier comprises a N-containing skeleton and a hydrophobic group connected to the N-containing skeleton. It is understandable that at least one H atoms connected with the N-atom of the N-containing skeleton is substituted with a hydrophobic group, such that the hydrophobic group is connected to the N-containing skeleton via a covalent bond. The present invention has a wide selection ranges for the N-containing skeleton, and preferably, the N-containing skeleton is provided by an amine compound comprising at least two amine groups, preferably at least one of diamine, triamine, tetramine and pentamine in a chain or ring form; more preferably, the amine compound is at least one selected from the group consisting of substituted or unsubstituted piperazine, ethylenediamine, propylenediamine, butanediamine, diethylene triamine, triethylene tetramine and tetraethylene pentamine. The substituent in the compound may be, for example, C1-C5 alkyl or alkylamino group.

In the case of an N-containing skeleton containing at least two amine groups, the hydrophobic groups are preferably connected with the N atoms at the ends of the N-containing skeleton, in the preferred case described above, the hydrophobic groups are concentrated outside the compound structure and have a synergistic action with the a hydrophilic N-containing skeleton in the compound, which is conducive to further improving the emulsifying efficiency of the composite emulsifier, and enhancing the electrical stability and the thermal stability of the emulsifying system.

According to the invention, the content of said N-containing skeleton is preferably within the range of 5-12 wt %, more preferably within the range of 7-10 wt %, based on the total amount of the composite emulsifier.

In the invention, a hydrophobic group refers to that the entire structural unit exhibits hydrophobicity (lipophilicity), and the specific composition of the hydrophobic group is not particularly limited in the invention. Preferably, the hydrophobic group contains at least one hydrophobic functional group. The invention has a wide selection range for the hydrophobic functional group, preferably, the hydrophobic functional group is at least one selected from the group consisting of alkyl, alkenyl, alkynyl, aryl and ester group. For example, the hydrophobic group may be straight-chain or branched C3-C18 alkyl, preferably any one of propyl, butyl, pentyl, hexyl, heptyl, nonyl, decyl, dodecyl, tetradecyl, hexadecyl, octadecyl, and isomers thereof, and any one of carbon atoms or hydrogen atoms of the alkyl may be further replaced by a substituent, for example, the substituent may be any one of ester group, hydroxyl and phenyl. For instance, the terminal carbon atom of the n-decyl may be substituted with a methyl ester group. The hydrophobic group may further contain a hydrophilic group such as hydroxyl, the hydrophilic group is not limited in the invention, as long as the structural unit exhibits hydrophobicity as a whole.

According to some preferred embodiments of the invention, the hydrophobic group is

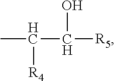

wherein R₄ is selected from H or C5-C16 alkyl; R₅ is selected from substituted or unsubstituted C5-C16 alkyl, alkoxy, aryl or phenoxy. The C5-C16 alkyl group is preferably any one of propyl, butyl, pentyl, hexyl, heptyl, nonyl, decyl, dodecyl, tetradecyl, hexadecyl, octadecyl, and isomers thereof, any one of carbon atoms or hydrogen atoms of the alkyl group may be further replaced by a substituent, for example, the substituent may be any one of ester group, hydroxyl and phenyl.

According to some preferred embodiments of the invention, the main emulsifier is a compound represented by formula (i) and/or formula (ii);

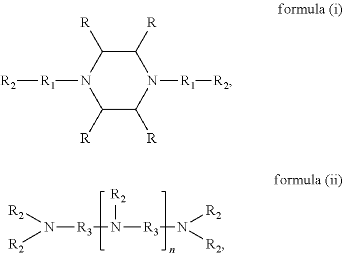

wherein in formula (i), each R is independently selected from H or C1-C3 alkyl, for example, it may be methyl, ethyl, n-propyl, or isopropyl; each R₁ is independently selected from

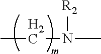

or absence, wherein m is a positive integer selected from 1-3; each R₂ is selected from H or the hydrophobic group, and the molar ratio of the hydrophobic group to N atom in said compound represented by formula (i) is (0.3-0.75):1, preferably (0.5-0.7):1.

In formula (ii), each R₂ is independently selected from H or the hydrophobic group, the molar ratio of the hydrophobic group to N atom is (0.2-1.2):1, preferably (0.4-1):1. Each R₃ is independently selected from substituted or unsubstituted C1-C3 alkylidene, which may be, for example, methylene, ethylene or propylene; n is a positive integer between 0 and 3, it may be, for instance, 0, 1, 2, 3; when n=0, R₃ is directly connected to the N atoms at both ends.

According to the invention, R₂ in the number n of repeated structural units in formula (ii) may be the same or different, each R₂ is independently selected from H or the hydrophobic group, as long as that the molar ratio of the hydrophobic group to the N atom satisfies the above requirements. Under the circumstance of the above molar ratio, the main emulsifier contains at least one primary amine and/or secondary amine structure, the primary amine and/or secondary amine structure has a relatively high sulfonation and/or carboxylation activity, such that an auxiliary emulsifier formed by sulfonation and/or carboxylation of the main emulsifier has an ideal structure, it is conducive to further enhancing the emulsification effect and stability of the obtained composite emulsifier.

According to a particular embodiment of the invention, when the N-containing skeleton is provided by triethylene tetramine, as shown in formula (ii), the structure of the main emulsifier may be represented by

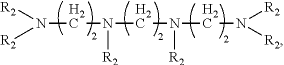

each R₂ is independently selected from H or the hydrophobic group, and the molar ratio of the hydrophobic group to N atoms is (0.2-1.2):1, i.e., it is understandable that R₂ may contain 1, 2, 3 or 4 hydrophobic groups, the remainder is H.

Another aspect of the invention provides a preparation method for the composite emulsifier, the preparation method comprises the following steps: contacting the main emulsifier with a sulfonating agent and/or a carboxylation reagent to introduce the sulfonic acid groups and/or carboxylic acid groups into a part of the main emulsifier.

In the invention, the structure of said main emulsifier has been described in detail in the preceding text, the content will not repeatedly described herein; the invention does not impose a specific requirements on the source of said main emulsifier, the main emulsifier can be prepared with any chemical reaction method known in the art. Preferably, the main emulsifier comprises a N-containing skeleton and a hydrophobic group connected to the N-containing skeleton, in the meanwhile, the preparation method of the main emulsifier may be, for example, performing a ring-opening reaction on an amine compound and an epoxy compound; the amine compound has the same definition as that in the preceding text.

Wherein the epoxy compound has a structure represented by formula (a),

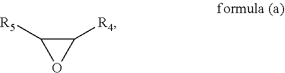

wherein R₄ and R₅ have the same definitions as those in the first aspect.

According to the invention, in step (1), the hydrophobic group is introduced into the amine compound by means of the ring-opening reaction of the amine compound with an epoxy compound.

According to some preferred embodiments of the invention, the epoxy compound is preferably at least one of methyl 9,10-epoxyoctadecanoate (cas: 6084-76-0), 1,2-epoxyoctadecane (cas: 7390-81-0) and C10-16-alkyl glycidyl ether (cas: 68081-84-5). The epoxy compound is commercially available, or may be prepared with a known method, the present invention does not impose the particular limitations thereto.

According to the invention, the molar ratio of the amine compound to the epoxy compound is preferably 1:(1-5), for example, it may be 1:1, 1:1.5, 1:2, 1:2.5, 1:3, 1:3.5, 1:4, 1:4.5, 1:5, and the other typical and non-limiting molar ratio or a random value within the range consisting of any two molar ratios. Preferably, the molar ratio of the amine compound to the epoxy compound is 1:(1.5-3.5).

In the present invention, an amine compound and an epoxy compound can be put into a reactor to carry out the ring-opening reaction. The conditions of the ring-opening reaction are not particularly limited in the invention, as long as the conditions may facilitate the ring-opening reaction of the epoxy compound, those skilled in the art can select the conditions according to actual needs. Preferably, the conditions of the ring-opening reaction comprise: the reaction temperature within the range of 60-140° C., more preferably within the range of 80-1200, and the reaction time within the range of 5-15 h, more preferably within the range of 6-12 h.

Preferably, the ring-opening reaction is carried out under the stirring conditions, and the stirring conditions are not particularly limited in the invention, as long as the reaction can be performed smoothly.

In the invention, the ring-opening reaction may be carried out in the presence of a catalyst, and the catalyst is not particularly limited in the invention, any catalyst known in the field that promotes the ring-opening reaction of an epoxy compound may be applied in the invention. For example, the catalyst may be an organic acid and/or an inorganic acid, preferably at least one of sulfuric acid, phosphoric acid, acetic acid, oxalic acid, trifluoroacetic acid, p-toluenesulfonic acid and sulfamic acid, further preferably at least one of sulfuric acid, phosphoric acid, acetic acid and oxalic acid.

Preferably, the molar ratio of the catalyst to the epoxy compound is (0.001-0.02):1, more preferably (0.003-0.015):1.

In the invention, the product of the ring-opening reaction may be directly used as the main emulsifier to continuously perform the next step reaction, or the product mixture obtained from the ring-opening reaction may be initially purified to remove unreacted raw materials which may be present by using various means, the next step reaction may be subsequently performed. The present invention has no special requirement, as long as the product contains the compound with the desired structure, those skilled in the art can make a choice according to the actual needs.

The invention has no special requirements on the specific type of the sulfonating agent, as long as the sulfonic acid group is introduced into a part of the main emulsifier, it can be selected by those skilled in the art according to the actual situation. The source of the sulfonating agent is not particularly limited in the invention, the sulfonating agent may be commercially available or prepared with the chemical reaction method known in the field.

According to some preferred embodiments of the invention, the sulfonating agent is sulfonate and/or alkyl sulfonate lactone.

Preferably, the sulfonate has a structure represented by formula (b),

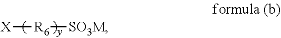

wherein X is a halogen atom, preferably C1 or Br; R₆ is selected from substituted or unsubstituted C1-C8 alkylidene; preferably C2-C4 alkylidene, for example, it may be any one of methylene, ethylidene, propylidene, butylidene, pentylidene, hexylidene, heptylidene and octylidene or isomers thereof; y is 0 or 1; M is selected from Na, K or ammonium group.

According to some preferred embodiments of the invention, the sulfonating agent is at least one selected from the group consisting of sodium 3-chloro-2-hydroxypropane sulfonate (cas: 126-83-0), 1,3-propane sultone (cas: 1120-71-4) and/or 1,4-butane sultone (cas: 1633-83-6), more preferably 1,3-propane sultone and/or 1,4-butane sultone.

The present application does not impose the specific requirements on the specific type of the carboxylation reagent, as long as the carboxylic acid groups can be introduced into a part of the main emulsifier, it can be selected by those skilled in the art according to the practical conditions; the source of the carboxylation reagent is not particularly defined in the invention either, it may be commercially available or prepared with the chemical reaction method known in the field. According to some preferred embodiments of the present invention, the carboxylation reagent is maleic anhydride.

According to the invention, in order to expedite the sulfonation reaction and/or the carboxylation reaction, and prevent the formation of an inorganic acid, the contacting is preferably carried out in the presence of an acid-binding agent. The specific type of the acid-binding agent is not particularly limited in the invention, it may be an acid-binding agent conventional in the field, for example, an organic alkali or an inorganic alkali, preferably an inorganic alkali, it may be, for instance, at least one of sodium carbonate, potassium carbonate, sodium hydroxide and potassium hydroxide.

According to the invention, the molar ratio of the acid-binding agent to the sulfonating agent and/or carboxylation reagent is preferably (0.5-2.5):1, more preferably (0.5-2):1. Under the preferable condition, the acid-binding agent is favorable for effectively binding the acidic byproduct generated in the reaction process and promoting the proceeding of the forward reaction, in the meanwhile, the acid-binding agent is alkaline salt, its addition in a suitable amount can effectively reduce the formation of salt content on the basis of not influencing the reaction, and avoid the defects that the reaction product has an overhigh viscosity and is difficult to pour and use on site.

According to the invention, the molar ratio of the total molar amount of the sulfonating agent and/or the carboxylation reagent to the main emulsifier calculated in terms of the N-containing skeleton is preferably (0.1-0.5):1, more preferably (0.2-0.4):1. A use of the above preferred embodiments allows to control the relative amount of the main emulsifier and the auxiliary emulsifier in the obtained composite emulsifier, which advantageously exert a synergistic effect of the main emulsifier and the auxiliary emulsifier, thereby further enhancing the emulsification effect of the emulsifier and improving the electrical stability and thermal stability of the emulsifying system.

The specific conditions under which the main emulsifier is contacted with a sulfonating agent and/or a carboxylation reagent are not particularly limited in the present invention, as long as the reaction can be carried out smoothly, those skilled in the art may select the conditions according to the actual needs. Preferably, the contacting conditions comprise: a reaction temperature within the range of 80-1200 and a time within the range of 3-5 hours.

In the invention, the contacting can also be carried out in the presence of a solvent, and the solvent may be selected from a wide range in the invention, and the solvent may be an organic solvent commonly used in the field, for example, at least one of methanol, ethanol, n-octanol, iso-octanol, dimethyl formamide, dimethyl sulfoxide and ethylene glycol. The dosage of the solvent is not particularly limited in the invention, as long as the reactants can be sufficiently dispersed, the solvent can be selected by those skilled in the art according to actual needs.

According to some preferred embodiments of the invention, the preparation method further comprises a step of removing the solvent from the mixture obtained by the reaction. In the present invention, the solvent in the product mixture may be removed by using various methods, preferably the reduced pressure distillation method. For example, a method for removing the solvent from the mixture obtained by the reaction comprises: cooling the mixture obtained by the reaction, and then carrying out the reduced pressure distillation. Preferably, the reduced pressure distillation is performed under a pressure of 1-5 kPa, more preferably 1.2-2 kPa, and a temperature within the range of 40-80° C., more preferably within the range of 45-60° C. . . . Unless otherwise specified in the invention, the pressure refers to an absolute pressure.

According to a particularly preferred embodiment of the invention, the preparation method for the composite emulsifier comprises the following steps:
(1) Subjecting an amine compound and an epoxy compound to the ring-opening reaction;
Wherein the epoxy compound has a structure represented by formula (a),

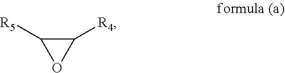

wherein R₄ and R₅ have the same definitions as those in the first aspect;
(2) Contacting the product of the ring-opening reaction with a sulfonating agent and/or a carboxylation reagent;
Wherein the ratio of the amine compound and the epoxy compound to the total molar amount of the sulfonating agent and/or the carboxylation reagent is 1:(1-5):(0.2-0.5).

According to the invention, a main emulsifier having an N-containing skeleton and a hydrophobic group connected to the skeleton is prepared by subjecting an amine compound and an epoxy compound to the ring-opening reaction, a sulfonic acid group and/or a carboxylic acid group are then introduced into a part of the main emulsifier by means of the sulfonation reaction and/or the carboxylation reaction, thereby obtaining the sulfonation product and/or carboxylation product of the main emulsifier, that is, a composite emulsifier comprising a main emulsifier and an auxiliary emulsifier is obtained. The composite emulsifier formed of the main emulsifier and the auxiliary emulsifier based on the same skeleton structure is more closely fused, the interaction of the main emulsifier and the auxiliary emulsifier can effectively reduce the oil-water interfacial tension, form a stable interfacial film, prevent the coalescence of water drops, and enable an oil-water emulsification system to become stable. The composite emulsifier serving as an oil-based drilling fluid emulsifier has excellent emulsification effect, desirable electrical stability and thermal stability, can be effectively applied in the oil-based drilling fluids having various oil-water ratios and different types of base oil, it is suitable for emulsification of oil-based drilling fluids such as gas oil, white oil and diesel.

Another aspect of the present invention provides a composite emulsifier, which is prepared with the raw materials comprising a polyamine material, epoxy fatty acid methyl ester, a catalyst, sodium 3-chloro-2-hydroxypropane sulfonate, an acid-binding agent;

Wherein the polyamine material is one or two selected from diethylene triamine and triethylene tetramine.

Preferably, the molar ratio of the polyamine material, epoxy fatty acid methyl ester, the catalyst, sodium 3-chloro-2-hydroxypropane sulfonate and the acid-binding agent is 1:(1.5-3.5):(0.005-0.02):(0.2-0.5):(0.2-0.5).

The second aspect of the present invention provides an oil-based drilling fluid comprising a base oil, and a treatment agent comprising the aforementioned composite emulsifier.

In the invention, the type of base oil in the oil-based drilling fluid can be selected from a wide range, it may be provided by oil phases conventionally adopted in the field, and it may be selected by those skilled in the art according to the actual needs. Preferably, the base oil is at least one selected from the group consisting of gas oil, white oil and diesel, more preferably white oil.

According to some preferred embodiments of the present invention, the composite emulsifier is contained in an amount of 2-10 parts by weight, more preferably 3-5 parts by weight, based on 100 parts by weight of the base oil.

According to the invention, for example, the treating agent may further comprise at least one of an organic soil, a plugging agent, a weighting agent, a wetting agent, an alkaline regulator and a filtrate reducer. Those skilled in the art can select the materials according to actual needs, and the invention does not impose specific limitations thereto.

According to some preferred embodiments of the invention, the organic soil may be at least one of organically modified bentonite, organically modified sepiolite, organically modified palygorskite, for example. The source of the organic soil in the invention is not particularly limited, it may be commercially available.

The source of the filtrate reducer is not particularly limited in the invention, the filtrate reducer may be the commercially available product well-known among those skilled in the art. Preferably, the filtrate reducer is at least one selected from the group consisting of natural asphalt, oxidized asphalt and styrene butadiene rubber.

According to some preferred embodiments of the invention, the weighting agent may be at least one of barite, micro-manganese ore powder and iron ore powder.

According to some preferred embodiments of the invention, the alkaline regulator may be calcium oxide and/or sodium carbonate, preferably calcium oxide.

In the invention, the dosages of the filtrate reducer, the alkaline regulator and the organic soil can be selected according to actual needs, and the dosages are not particularly limited in the invention. According to some preferred embodiments of the invention, the dosage of the filtrate reducer is 2-6 parts by weight, the dosage of the alkaline regulator is 0.5-2 parts by weight, and the dosage of the organic soil is 0.5-3 parts by weight, based on 100 parts by weight of the base oil.

The present invention will be described in detail below with reference to examples.

Unless otherwise specified in the following examples and comparative examples, the raw materials in use were all commercially available.

In the invention, the method for testing the demulsification voltage and the emulsification rate of a composite emulsifier in the standard oil-based emulsion comprised the following steps: the composite emulsifier and 5 #white oil were stirred at high speed for 30 minutes under the condition of 10,000 r/min to dissolve the emulsifier in the oil, the CaCl₂) aqueous solution with a concentration of 25 wt % was then added according to the oil-water volume ratio of 80:20, and stirred at a high speed of 10,000 r/min for 2 h, the emulsion to be detected was obtained. The addition amount of the composite emulsifier was 5 wt %, based on the total mass of the oil and the water.

The electrical stability test: a probe of an electrical stability tester was placed in the emulsion to be tested, the measurement results were recorded, the average demulsification voltage value of the two measurement results were calculated, wherein the difference of the two readings was not more than 5%.

The emulsification rate test: the emulsion to be detected was subjected to an aging process at 1500 for 16 h, then cooled to room temperature, stirred at a high speed of 10,000 r/min for 20 min, the emulsion was poured into a 500 mL measuring cylinder, subjected to standing still for 24 h, and the volume of the separated oil phase was read. The emulsification rate (W) was calculated according to the following equation:

$$W = ( {V}_{0} - V ) / {V}_{0} \times 100 \% ;$$

In the above formula, V₀ denoted the volume of an emulsion, and the unit was mL; W denoted the emulsification rate, the unit was %; V denoted the volume of separated oil layer, and the unit was mL.

In the invention, an organic element analyzer with an instrument model Elementar Unicube was used for measuring the contents of C, H, O, N, S elements in the product. The molar ratio of the S element and the N element in the emulsifier was obtained by further calculation.

In the invention, the infrared absorption spectrum was tested by a Fourier infrared spectrometer, and the test mode was an Attenuated Total Reflectance (ATR) mode.

In the present invention, the mass spectrometry was tested in a matrix-assisted laser desorption ionization time-of-flight mass spectrometry (MALDI-TOF/TOF), and the test conditions comprised: dissolved with methanol, the matrix was CHCA (Cyano-4-hydroxycinnamic acid).

### Example 1

(1) 20 mol of diethylene triamine and 38 mol of methyl 9,10-epoxyoctadecanoate (cas: 6084-76-0, commercially available, with the purity of 75%) were added into a double-neck round-bottom flask with a reflux condenser pipe, the materials were mixed and stirred uniformly, 0.2 mol of para-toluenesulfonic acid was added and blended, and then stirred and heated to 90° C. for performing the reaction for 7.5 hours, a main emulsifier was obtained.

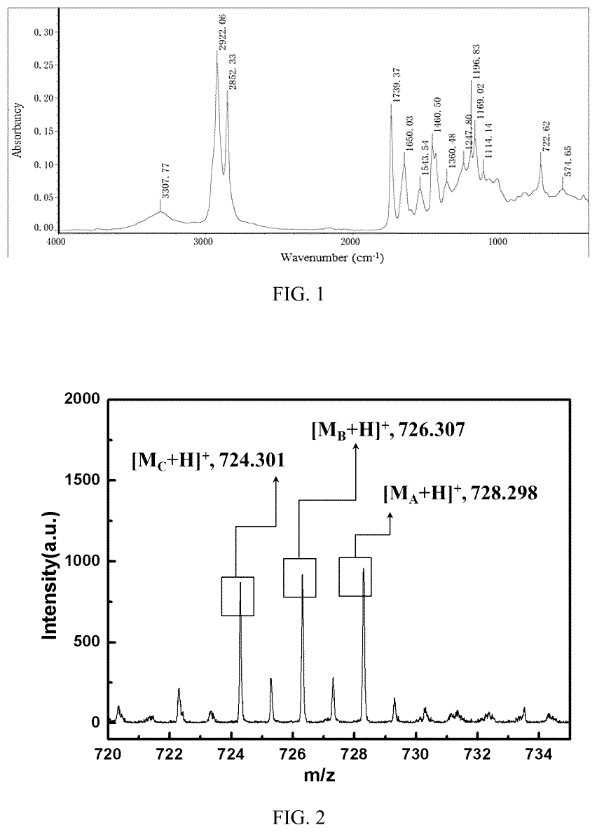

A part of main emulsifier was taken and separated by the silica gel column chromatography (the ratio of petroleum ether to ethyl acetate was 2:1-1:5), a solvent was distilled out by a rotary evaporator to obtain a purified product, which was then subjected to an infrared absorption spectrum analysis, the results were shown in FIG. 1, the infrared absorption spectrum showed that the absorption peak belonging to epoxy bonds at 1080-1160 cm⁻¹ was disappeared, the characteristic peak belonging to hydroxyl groups at 3200-3500 cm⁻¹ was very obvious, it demonstrated that the ring-opening reaction of diethylene triamine and epoxy fatty acid methyl ester was carried out. The mass spectrometry was performed on the main emulsifier, the results were shown in FIG. 2, the structure corresponding to characteristic peaks with the mass-to-charge ratios of 724.301, 726.307 and 728.298 was diethylene triamine substituted by dihydroxy fatty acid methyl ester, the multimodal signals were derived from the presence of linoleic acid methyl ester and oleic acid methyl ester in the methyl 9,10-epoxyoctadecanoate component, the epoxy methyl oleate and epoxy methyl linoleate can be generated after epoxidation of linoleic acid methyl ester and oleic acid methyl ester. The above results proved that the main emulsifier was successfully synthesized, it was demonstrated with reference to the results of mass spectrometry and infrared adsorption spectrum that the main emulsifier had a structure represented by formula (i), wherein two groups R₂ were

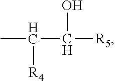

R₄ was n-octyl carbomethoxy, and R₅ was n-octyl. The molar ratio of the hydrophobic group to N atoms in the diethylene triamine skeleton was 2:3.

(2) The product obtained in step (1), 6 mol of sodium 3-chloro-2-hydroxy propane sulfonate and 150 mL of ethanol were uniformly mixed, 6 mol of potassium carbonate was added and mixed, the mixture was heated to 80° C., stirred and subjected to the reaction for 3 hours, a second intermediate was obtained;

(3) The second intermediate obtained in step (2) was cooled to room temperature, then reduced pressure distilled under the pressure of 1.5 kPa and the temperature of 500 to remove solvent ethanol, a composite emulsifier A1 was prepared.

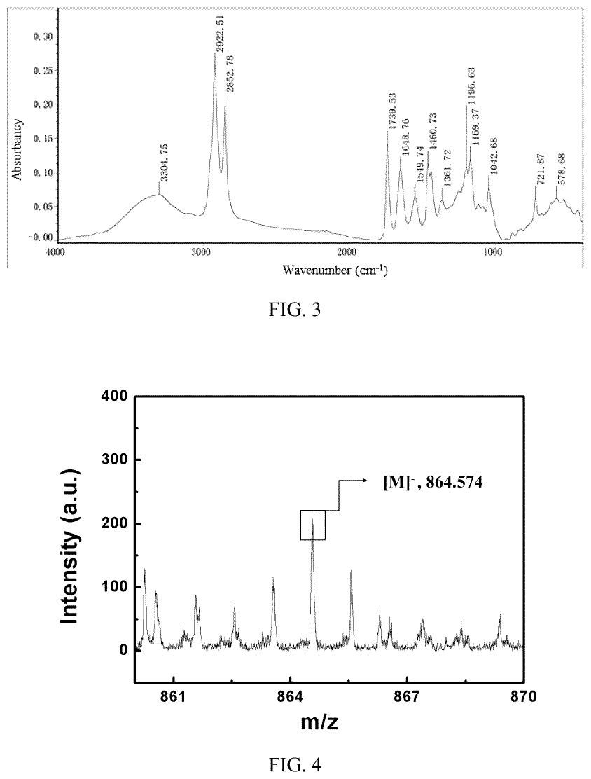

The composite emulsifier A1 was separated by the silica gel column chromatography (the ratio of dichloromethane to methanol was 1:1-1:8), the solvent was distilled out by a rotary evaporator to obtain a purified product, which was then subjected to an infrared absorption spectrum test, the infrared absorption spectrum was shown in FIG. 3, the infrared absorption spectrum showed that the characteristic absorption peak belonging to hydroxyl at 3200-3500 cm⁻¹ became large, which indicated that the content of hydroxyl was increased, and the characteristic peak belonging to sulfonate group at 1042.68 cm⁻¹ appeared, which demonstrated that sulfonate group existed in the structure. The mass spectrometry results were shown in FIG. 4, the structure corresponding to the characteristic peak with the mass-to-charge ratio of 864.574 was diethylene triamine substituted by the dihydroxy fatty acid methyl ester into which a sulfonic acid group was introduced, it was indicated with reference to the results of FIG. 3 and FIG. 4 that a sulfonic acid group was introduced into diethylene triamine skeleton of the main emulsifier. The elemental composition was analyzed by an organic element analyzer, wherein the content of N element was 4.92 wt %, the content of C element was 65.4 wt %, the content of H element was 10 wt %, the content of S element was 1.15 wt %, and the content of O element was 16.95 wt %. The calculated molar ratio of the auxiliary emulsifier to the main emulsifier was 0.44:1.

The content of the diethylene triamine skeleton was 12 wt % based on the total amount of the composite emulsifier. The molar amount of sulfonic acid groups in 1 mol of the composite emulsifier A1 was 0.31 mol.

The emulsification rate and demulsification voltage of the composite emulsifier A1 in standard oil-based emulsions were shown in Table 1.

### Example 2

(1) 20 mol of diethylene triamine and 38 mol of methyl 9,10-epoxyoctadecanoate (cas: 6084-76-0) were added into a double-neck round-bottom flask with a reflux condenser pipe, the materials were mixed and stirred uniformly, 0.25 mol of sulfuric acid was added and blended, and then stirred and heated to 1000 for performing the reaction for 8 hours, a main emulsifier was obtained. The molar ratio of the hydrophobic group to N atoms in the diethylene triamine skeleton was 2:3.

(2) The main emulsifier, 6 mol of sodium 3-chloro-2-hydroxy propane sulfonate and 200 mL of isooctanol were uniformly mixed, 6 mol of sodium hydroxide was added and mixed, the mixture was heated to 900, stirred and subjected to the reaction for 4 hours;

(3) The product obtained in step (2) was cooled to room temperature, then reduced pressure distilled under the pressure of 1.2 kPa and the temperature of 50° C. to remove solvent isooctanol, a composite emulsifier A2 was prepared.

The composite emulsifier A2 was separated by the silica gel column chromatography (the ratio of dichloromethane to methanol was 1:1-1:8), the solvent was distilled out by a rotary evaporator to obtain a purified product, the elemental composition was then analyzed by an organic element analyzer, wherein the content of N element was 5.1 wt %, the content of C element was 64.2 wt %, the content of H element was 11.2 wt %, the content of S element was 1.2 wt %, and the content of O element was 16.7 wt %. The calculated molar ratio of the auxiliary emulsifier to the main emulsifier was 0.45:1. The molar ratio of S element to N element in the emulsifier was 0.1:1. The content of the diethylene triamine skeleton was 12.5 wt %, based on the total amount of the emulsifier. The molar amount of sulfonic acid groups in 1 mol of the composite emulsifier A2 was 0.31 mol.

The emulsification rate and demulsification voltage of the composite emulsifier A2 in standard oil-based emulsions were shown in Table 1.

### Example 3

(1) 20 mol of triethylene tetramine and 45 mol of methyl 9,10-epoxyoctadecanoate (cas: 6084-76-0) were added into a double-neck round-bottom flask with a reflux condenser pipe, the materials were mixed and stirred uniformly, 0.3 mol of phosphoric acid was added and blended, and then stirred and heated to 80° C. for performing the reaction for 6 hours, a main emulsifier was obtained. The molar ratio of the hydrophobic group to N atoms in triethylene tetramine skeleton was 0.56:1.

(2) The main emulsifier, 8 mol of sodium 3-chloro-2-hydroxy propane sulfonate and 150 mL of isopropanol were uniformly mixed, 4 mol of potassium carbonate was added and mixed, the mixture was heated to 80° C., stirred and subjected to reaction for 3 hours, a second intermediate was obtained;

(3) The second intermediate was cooled to room temperature, then reduced pressure distilled under the pressure of 1.2 kPa and the temperature of 500 to remove solvent isopropanol, a composite emulsifier A3 was prepared.

The composite emulsifier A3 was separated by the silica gel column chromatography (the ratio of dichloromethane to methanol was 1:1-1:8), the solvent was distilled out by a rotary evaporator to obtain a purified product, the elemental composition was then analyzed by an organic element analyzer, wherein the content of N element was 4.3 wt %, the content of C element was 64.4 wt %, the content of H element was 10.7 wt %, the content of S element was 1.1 wt %, and the content of O element was 15.9 wt %. The calculated molar ratio of the auxiliary emulsifier to the main emulsifier was 0.75:1.

The content of the triethylene tetramine skeleton was 11.2 wt %, based on the total amount of the composite emulsifier. The molar amount of sulfonic acid groups in 1 mol of the composite emulsifier A3 was 0.45 mol.

The emulsification rate and demulsification voltage of the composite emulsifier A3 in standard oil-based emulsions were shown in Table 1.

### Example 4

(1) 20 mol of triethylene tetramine and 52 mol of methyl 9,10-epoxyoctadecanoate (cas: 6084-76-0) were added into a double-neck round-bottom flask with a reflux condenser pipe, the materials were mixed and stirred uniformly, 0.2 mol of para-toluenesulfonic acid was added and blended, and then stirred and heated to 110° C. for performing the reaction for 12 hours, a first intermediate was obtained. The molar ratio of the hydrophobic group to N atoms in the triethylene tetramine skeleton was 0.65:1.

(2) The first intermediate, 8 mol of sodium 3-chloro-2-hydroxy propane sulfonate and 300 mL of n-octanol were uniformly mixed, 8 mol of potassium carbonate was added and mixed, the mixture was heated to 1200, stirred and subjected to reaction for 5 hours, a second intermediate was obtained;

(3) The second intermediate was cooled to room temperature, then reduced pressure distilled under the pressure of 1.2 kPa and the temperature of 50° C. to remove solvent n-octanol, a composite emulsifier A4 was prepared.

The composite emulsifier A4 was separated by the silica gel column chromatography (the ratio of dichloromethane to methanol was 1:1-1:8), the solvent was distilled out by a rotary evaporator to obtain a purified product, the elemental composition was then analyzed by an organic element analyzer, wherein the content of N element was 4.0 wt %, the content of C element was 65.4 wt %, the content of H element was 11.4 wt %, the content of S element was 0.9 wt %, and the content of O element was 16.8 wt %. The calculated molar ratio of the auxiliary emulsifier to the main emulsifier was 0.65:1.

The content of the triethylene tetramine skeleton was 10 wt %, based on the total amount of the composite emulsifier. The molar amount of sulfonic acid groups in 1 mol of the composite emulsifier was 0.39 mol.

The emulsification rate and demulsification voltage of the composite emulsifier A4 in standard oil-based emulsions were shown in Table 1.

### Example 5

The composite emulsifier was prepared according to the method in Example 1, except that the methyl 9,10-epoxyoctadecanoate (cas: 6084-76-0) was replaced with an equimolar amount of 1,2-epoxy dodecane, a composite emulsifier A5 was obtained.

The composite emulsifier A5 was separated by the silica gel column chromatography (the ratio of dichloromethane to methanol was 1:1-1:8), the solvent was distilled out by a rotary evaporator to obtain a purified product, the elemental composition was then analyzed by an organic element analyzer, wherein the content of N element was 5.9 wt %, the content of C element was 67.3 wt %, the content of H element was 11.9 wt %, the content of S element was 1.3 wt %, and the content of O element was 8.9 wt %. The calculated molar ratio of the auxiliary emulsifier to the main emulsifier was 0.41:1.

The content of the triethylene tetramine skeleton was 14.5 wt %, based on the total amount of the composite emulsifier. The molar amount of sulfonic acid groups in 1 mol of the composite emulsifier was 0.29 mol.

The emulsification rate and demulsification voltage of the composite emulsifier A5 in standard oil-based emulsions were shown in Table 1.

### Example 6

The composite emulsifier was prepared according to the method in Example 1, except that the sodium 3-chloro-2-hydroxypropane sulfonate was replaced with an equimolar amount of 1,3-propane sultone, a composite emulsifier A6 was obtained.

The composite emulsifier A6 was separated by the silica gel column chromatography (the ratio of dichloromethane to methanol was 1:1-1:8), the solvent was distilled out by a rotary evaporator to obtain a purified product, the elemental composition was then analyzed by an organic element analyzer, wherein the content of N element was 5.4 wt %, the content of C element was 66.1 wt %, the content of H element was 10.8 wt %, the content of S element was 1.3 wt %, and the content of O element was 15.1 wt %. The calculated molar ratio of the auxiliary emulsifier to the main emulsifier was 0.47:1.

The content of the diethylene triamine skeleton was 13.2 wt %, based on the total amount of the emulsifier. The molar amount of sulfonic acid groups in 1 mol of the composite emulsifier was 0.32 mol. The emulsification rate and demulsification voltage of the composite emulsifier A6 in standard oil-based emulsions were shown in Table 1.

### Example 7

The composite emulsifier was prepared according to the method in Example 1, except that the sodium 3-chloro-2-hydroxypropane sulfonate was used in an amount of 14 mol, a composite emulsifier A7 was obtained.

The composite emulsifier A7 was separated by the silica gel column chromatography (the ratio of dichloromethane to methanol was 1:1-1:8), the solvent was distilled out by a rotary evaporator to obtain a purified product, the elemental composition was then analyzed by an organic element analyzer, wherein the content of N element was 5.0 wt %, the content of S element was 2.73 wt %. The calculated molar ratio of the auxiliary emulsifier to the main emulsifier was 2.5:1.

The content of the diethylene triamine skeleton was 11.9 wt %, based on the total amount of the emulsifier. The molar amount of sulfonic acid groups in 1 mol of the composite emulsifier was 0.71 mol.

The emulsification rate and demulsification voltage of the emulsifier A7 in standard oil-based emulsions were shown in Table 1.

### Example 8

The composite emulsifier was prepared according to the method in Example 1, except that the sodium 3-chloro-2-hydroxypropane sulfonate was used in an amount of 4.2 mol, a composite emulsifier A8 was obtained.

The composite emulsifier A8 was separated by the silica gel column chromatography (the ratio of dichloromethane to methanol was 1:1-1:8), the solvent was distilled out by a rotary evaporator to obtain a purified product, the elemental composition was then analyzed by an organic element analyzer, wherein the content of N element was 5.6 wt %, the content of S element was 0.8 wt %. The calculated molar ratio of the auxiliary emulsifier to the main emulsifier was 0.23:1.

The content of the diethylene triamine skeleton was 13.3 wt %, based on the total amount of the emulsifier. The molar amount of sulfonic acid groups in 1 mol of the composite emulsifier was 0.19 mol.

The emulsification rate and demulsification voltage of the composite emulsifier A8 in standard oil-based emulsions were shown in Table 1.

### Example 9

(1) 20 mol of aminoethyl piperazine and 38 mol of methyl 9,10-epoxyoctadecanoate (cas: 6084-76-0) were added into a double-neck round-bottom flask with a reflux condenser pipe, the materials were mixed and stirred uniformly, 0.25 mol of para-toluenesulfonic acid was added and blended, and then stirred and heated to 100° C. for performing the reaction for 8 hours, a main emulsifier was obtained. The molar ratio of the hydrophobic group to N atoms in the aminoethyl piperazine skeleton was 2:3.
(2) The main emulsifier, 6 mol of sodium 3-chloro-2-hydroxy propane sulfonate and 200 mL of isooctanol were uniformly mixed, 6 mol of sodium hydroxide was added and mixed, the mixture was heated to 900, stirred and subjected to reaction for 4 hours;
(3) The product obtained in step (2) was cooled to room temperature, then reduced pressure distilled under the pressure of 1.2 kPa and the temperature of 50° C. to remove solvent isooctylol, a composite emulsifier A2 was prepared.

The composite emulsifier A2 was separated by the silica gel column chromatography (the ratio of dichloromethane to methanol was 1:1-1:8), the solvent was distilled out by a rotary evaporator to obtain a purified product, the elemental composition was then analyzed by an organic element analyzer, wherein the content of N element was 5.1 wt %, the content of C element was 63.2 wt %, the content of H element was 10.4 wt %, the content of S element was 1.1 wt %, and the content of O element was 15.2 wt %. The calculated molar ratio of the auxiliary emulsifier to the main emulsifier was 0.38:1. The molar ratio of S element to N element in the emulsifier was 0.09:1. The content of the aminoethyl piperazine skeleton was 12.51 wt %, based on the total amount of the emulsifier. The molar amount of sulfonic acid groups in 1 mol of the composite emulsifier A2 was 0.28 mol.

The emulsification rate and demulsification voltage of the composite emulsifier A9 in standard oil-based emulsions were shown in Table 1.

### Example 10

(1) 20 mol of ethylene diamine and 38 mol of methyl 9,10-epoxyoctadecanoate (cas: 6084-76-0) were added into a double-neck round-bottom flask with a reflux condenser pipe, the materials were mixed and stirred uniformly, 0.25 mol of para-toluenesulfonic acid was added and blended, and then stirred and heated to 1000 for performing the reaction for 8 hours, a main emulsifier was obtained. The molar ratio of the hydrophobic group to N atoms in the ethylene diamine skeleton was 1:1.
(2) The main emulsifier, 6 mol of sodium 3-chloro-2-hydroxy propane sulfonate and 200 mL of isooctanol were uniformly mixed, 6 mol of sodium hydroxide was added and mixed, the mixture was heated to 900, stirred and subjected to the reaction for 4 hours;
(3) The product obtained in step (2) was cooled to room temperature, then reduced pressure distilled under the pressure of 1.2 kPa and the temperature of 50° C. to remove solvent isooctanol, a composite emulsifier A2 was prepared.

The composite emulsifier A2 was separated by the silica gel column chromatography (the ratio of dichloromethane to methanol was 1:1-1:8), the solvent was distilled out by a rotary evaporator to obtain a purified product, the elemental composition was then analyzed by an organic element analyzer, wherein the content of N element was 3.7 wt %, the content of S element was 1.3 wt %. The calculated molar ratio of the auxiliary emulsifier to the main emulsifier was 0.43:1. The molar ratio of S element to N element in the emulsifier was 0.154:1. The content of the ethylene diamine skeleton was 7.9 wt %, based on the total amount of the emulsifier. The molar amount of sulfonic acid groups in 1 mol of the composite emulsifier A2 was 0.30 mol.

The emulsification rate and demulsification voltage of the composite emulsifier A10 in standard oil-based emulsions were shown in Table 1.

### Comparative Example 1

The emulsifier was prepared according to the method in Example 1, except that steps (2) and (3) were not performed, the main emulsifier obtained in step (1) was used as an emulsifier DA1.

The emulsification rate and demulsification voltage of the emulsifier DA1 in standard oil-based emulsions were shown in Table 1.

### Comparative Example 2

(1) 20 mol of diethylene triamine and 38 mol of methyl 9,10-epoxyoctadecanoate (cas: 6084-76-0) were added into a double-neck round-bottom flask with a reflux condenser pipe, the materials were mixed and stirred uniformly, 0.2 mol of para-toluenesulfonic acid was added and blended, and then stirred and heated to 90° C. for performing the reaction for 7.5 hours, a first intermediate was obtained;
(2) The first intermediate, 22 mol of sodium 3-chloro-2-hydroxy propane sulfonate and 1,000 mL of n-octanol were uniformly mixed, 22 mol of potassium carbonate was added and mixed, the mixture was heated to 1000, stirred and subjected to reaction for 4 hours, a second intermediate was obtained;
(3) The second intermediate was cooled to room temperature, then reduced pressure distilled under the pressure of 1.2 kPa and the temperature of 50° C. to remove solvent ethanol, a composite emulsifier DA2 was prepared.

The composite emulsifier DA2 was separated by the silica gel column chromatography (the ratio of dichloromethane to methanol was 1:1-1:8), the solvent was distilled out by a rotary evaporator to obtain a purified product, the elemental composition was then analyzed by an organic element analyzer, wherein the content of N element was 4.1 wt %, the content of S element was 3.4 wt %. The emulsifier contained only the auxiliary emulsifier. The content of the diethylene triamine skeleton was 10 wt %, based on the total amount of the composite emulsifier. The molar amount of sulfonic acid groups in 1 mol of the composite emulsifier was 1.09 mol.

The emulsification rate and demulsification voltage of the composite emulsifier DA2 in standard oil-based emulsions were shown in Table 1.

### Comparative Example 3

(1) 20 mol of triethylene tetramine and 40 mol of methyl 9,10-epoxyoctadecanoate (cas: 6084-76-0) were added into a double-neck round-bottom flask with a reflux condenser pipe, the materials were mixed and stirred uniformly, 0.2 mol of para-toluenesulfonic acid was added and blended, and then stirred and heated to 80° C. for performing the reaction for 6 hours, a first intermediate was obtained.
(2) The first intermediate, 40 mol of sodium 3-chloro-2-hydroxy propane sulfonate and 2,000 mL of n-octanol were uniformly mixed, 40 mol of potassium carbonate was added and mixed, the mixture was heated to 1200, stirred and subjected to reaction for 8 hours, a second intermediate was obtained;
(3) The second intermediate was cooled to room temperature, then reduced pressure distilled under the pressure of 1.2 kPa and the temperature of 50° C. to remove solvent n-octanol, an emulsifier DA3 was prepared.

The emulsifier DA3 was separated by the silica gel column chromatography (the ratio of dichloromethane to methanol was 1:1-1:8), the solvent was distilled out by a rotary evaporator to obtain a purified product, the elemental composition was then analyzed by an organic element analyzer, wherein the content of N element was 5.1 wt %, the content of S element was 5.9 wt %. The molar ratio of S element to N element in the emulsifier was 1.5:1. The content of the triethylene tetramine skeleton was 13.3 wt % based on the total amount of the emulsifier. The emulsifier contained only the auxiliary emulsifier. The molar amount of sulfonic acid groups in 1 mol of the composite emulsifier was 2.02 mol.

The emulsification rate and demulsification voltage of the composite emulsifier DA3 in standard oil-based emulsions were shown in Table 1.

### Comparative Example 4

The main emulsifier Span 80 was mixed with the auxiliary emulsifier Tween 80 at a molar ratio 3:1, the mixture was used as an emulsifier DA4.

The emulsification rate and demulsification voltage of the composite emulsifier DA4 in standard oil-based emulsions were shown in Table 1.

TABLE 1

| | | Volume | Addition | | |
|---|---|---|---|---|---|
| Emulsifier | | ratio of | amount of the | Emulsification | Demulsification |
| Number | Base oil | oil-water | emulsifier wt % | ratio % | voltage V |
| A1 | 5# white oil | 80:20 | 5 | 100 | 264 |
| A1 | 5# white oil | 80:20 | 5 | 100 | 274 |
| purified | | | | | |
| product | | | | | |
| A2 | 5# white oil | 80:20 | 5 | 100 | 272 |
| A3 | 5# white oil | 80:20 | 5 | 100 | 245 |
| A4 | 5# white oil | 80:20 | 5 | 100 | 238 |
| A5 | 5# white oil | 80:20 | 5 | 100 | 212 |
| A6 | 5# white oil | 80:20 | 5 | 100 | 227 |
| A7 | 5# white oil | 80:20 | 5 | 92 | 125 |
| A8 | 5# white oil | 80:20 | 5 | 100 | 389 |
| A9 | 5# white oil | 80:20 | 5 | 100 | 227 |
| A10 | 5# white oil | 80:20 | 5 | 100 | 211 |
| DA1 | 5# white oil | 80:20 | 5 | 100 | 432 |
| DA2 | 5# white oil | 80:20 | 5 | 53 | 54 |
| DA3 | 5# white oil | 80:20 | 5 | 48 | 47 |
| DA4 | 5# white oil | 80:20 | 5 | 24 | 384 |

### Test Example

### Preparation of Drilling Fluids:

Based on 100 parts by weight of 5 #white oil, 4 parts by weight of the emulsifiers prepared in the Examples and Comparative Example were added separately, the materials were stirred at high speed of 10,000 rpm for 30 min, the CaCl₂) aqueous solution with a concentration of 25 wt % was then added according to the volume ratio of 80:20. Based on 100 parts by weight of 5 #white oil, 4 parts by weight of natural asphalt, 2 parts by weight of calcium oxide, 2 parts by weight of organic bentonite were subsequently added, barite was then added to improve the weight and increase the density to the range of 1.8-2.2 g/cm³, the stirring with a high speed was continued for 2 h, the oil-based drilling fluids with a density of 2 g/cm3 were prepared.
(1) The electrical stability test: a probe of an electrical stability tester was placed in the prepared oil-based drilling fluid, the measurement results were recorded, the average demulsification voltage value of the two measurement results were calculated, wherein the difference of the two readings was not more than 5%.

The plastic viscosity (PV), yield point (YP) and high-temperature high-pressure filtrate loss (FLHTHP) of the drilling fluids were tested according to the China National Standard GB/T16783.2-2012 “Petroleum and natural gas industries-Field testing of drilling fluids-Part 2: Oil-based fluids”. The results were shown in Table 2.

TABLE 2

| | Density | | | | Demulsification |
|---|---|---|---|---|---|
| No. | g/cm³ | PV/mPs | YP/Pa | FLHTHP/mL | voltage/V |
| A1 | 2 | 33 | 5.5 | 2.2 | 970 |
| A1 | 2 | 34 | 5.5 | 1.8 | 1089 |
| purified | | | | | |
| product | | | | | |
| A2 | 1.8 | 26 | 4 | 2.2 | 1014 |
| A3 | 2.2 | 39 | 5.5 | 2.6 | 846 |
| A4 | 2 | 32 | 5.5 | 2.4 | 912 |
| A5 | 2 | 35 | 5 | 3 | 712 |
| A6 | 2 | 36 | 4.5 | 3.2 | 689 |
| A7 | 2 | 31 | 5 | 2.6 | 674 |
| A8 | 2 | 38 | 6.5 | 2.8 | 742 |
| A9 | 2 | 34 | 5.5 | 2.6 | 867 |
| A10 | 2 | 36 | 6 | 2.4 | 794 |
| DA1 | 2 | 40 | 7 | 4.2 | 501 |
| DA2 | 2 | 30 | 4 | 5.8 | 524 |
| DA3 | 2 | 28 | 4.5 | 6.4 | 572 |
| DA4 | 2 | 38 | 5.5 | 4.4 | 567 |

(2) Thermal stability test

The prepared drilling fluids were subjected to an aging process at 1800 for 24 h, respectively, the drilling fluid properties were tested according to the same method, the results were shown in Table 3.

The prepared drilling fluids were subjected to an aging process at 2000 for 96 h, respectively, the drilling fluid properties were tested according to the same method, the results were shown in Table 4.

TABLE 3
After the aging process (180° C./24 h)

| | Density | | | | Demulsification |
|---|---|---|---|---|---|
| No. | g/cm³ | PV/mPs | YP/Pa | FLHTHP/mL | voltage/V |
| A1 | 2 | 26 | 4.5 | 2.8 | 828 |
| A1 | 2 | 25 | 5 | 2.2 | 974 |
| purified | | | | | |
| product | | | | | |
| A2 | 1.8 | 23 | 3.5 | 2.8 | 932 |
| A3 | 2.2 | 35 | 4 | 3 | 725 |
| A4 | 2 | 31 | 5 | 3.4 | 742 |
| A5 | 2 | 24 | 3.5 | 3.6 | 611 |
| A6 | 2 | 27 | 3 | 3.8 | 572 |
| A7 | 2 | 24 | 4 | 2.4 | 691 |
| A8 | 2 | 42 | 7 | 3.2 | 518 |
| A9 | 2 | 25 | 4.5 | 3.0 | 724 |
| A10 | 2 | 26 | 4.0 | 3.6 | 698 |
| DA1 | 2 | 52 | 8.5 | 6.2 | 368 |
| DA2 | 2 | 20 | 2 | 6.8 | 478 |
| DA3 | 2 | 22 | 2 | 7.2 | 420 |

TABLE 4
After the aging process (200° C./96 h)

| | Density | | | | Demulsification |
|---|---|---|---|---|---|
| No. | g/cm³ | PV/mPs | YP/Pa | FLHTHP/mL | voltage/V |
| A1 | 2 | 26 | 2 | 4.2 | 546 |
| A1 | 2 | 24 | 3.5 | 3.2 | 648 |
| purified | | | | | |
| product | | | | | |
| A2 | 1.8 | 23 | 1.5 | 4.8 | 512 |
| A3 | 2.2 | 24 | 2 | 4.4 | 504 |
| A4 | 2 | 24 | 1.5 | 4.4 | 508 |
| A5 | 2 | 24 | 1.5 | 4.8 | 432 |
| A6 | 2 | 26 | 2 | 4.6 | 464 |
| A7 | 2 | 24 | 1.5 | 4.6 | 432 |
| A8 | 2 | 52 | 3.5 | 8.4 | 437 |
| A9 | 2 | 23 | 2 | 4.2 | 546 |
| A10 | 2 | 25 | 1.5 | 4.8 | 501 |
| DA1 | 2 | 62 | 10.5 | 12.4 | 312 |
| DA2 | 2 | 16 | 0 | 14.8 | 348 |
| DA3 | 2 | 18 | 0 | 16.8 | 291 |

As can be seen with reference to the results in Tables 2-4, the composite emulsifier products prepared in Examples of the present invention have superior electrical stability and thermal stability than the emulsifiers prepared in Comparative Examples, and maintain obvious advantages such as a low high-temperature and high-pressure filtrate loss.

The above content describes in detail the preferred embodiments of the present disclosure, but the present disclosure is not limited thereto. A variety of simple modifications can be made in regard to the technical solutions of the present disclosure within the scope of the technical concept of the present disclosure, including a combination of individual technical features in any other suitable manner, such simple modifications and combinations thereof shall also be regarded as the content disclosed by the present disclosure, each of them falls into the protection scope of the present disclosure.

1.-10. (canceled)

11. A composite emulsifier comprising a main emulsifier and an auxiliary emulsifier, wherein the auxiliary emulsifier is a sulfonated substance and/or carboxylation product of the main emulsifier.

12. The composite emulsifier according to claim 11, wherein the demulsification voltage of the composite emulsifier in a standard oil-based emulsion under the condition of a dosage of 5 wt % is within the range of 50-400V, wherein the standard oil-based emulsion is composed of 5 #white oil and CaCl₂) aqueous solution having a concentration of 25 wt %, the volume ratio of oil-water is 80:20.

13. The composite emulsifier according to claim 11, the emulsification ratio of the composite emulsifier in the standard oil-based emulsion is larger than or equal to 90%.

14. The composite emulsifier according to claim 11, wherein the molar ratio of the auxiliary emulsifier to the main emulsifier in the composite emulsifier is (0.25-1):1.

15. The composite emulsifier according to 11, wherein the auxiliary emulsifier contains sulfonic acid groups and/or carboxylic acid groups, wherein a molar amount of the sulfonic acid groups and/or carboxylic acid groups in 1 mol of the composite emulsifier is within the range of 0.1-1.2 mol.

16. The composite emulsifier according to claim 11, wherein the main emulsifier comprises a N-containing skeleton and a hydrophobic group connected to the N-containing skeleton.

17. The composite emulsifier according to claim 16, wherein the content of the N-containing skeleton is within the range of 5-20 wt %, based on the total amount of the composite emulsifier.

18. The composite emulsifier according to claim 16, wherein the N-containing skeleton is provided by an amine compound comprising at least two amine groups.

19. The composite emulsifier according to claim 18, wherein the amine compound is at least one selected from the group consisting of substituted or unsubstituted piperazine, ethylenediamine, propylenediamine, butanediamine, diethylene triamine, triethylene tetramine and tetraethylene pentamine.

20. The composite emulsifier according to claim 16, wherein the hydrophobic group comprises at least one hydrophobic functional group; and/or, the hydrophobic group is represented by 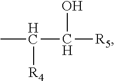
wherein R₄ is selected from H or C5-C16 alkyl; and R₅ is selected from substituted or unsubstituted C5-C16 alkyl, alkoxy, aryl or phenoxy.

21. The composite emulsifier according to claim 16, wherein the main emulsifier is a compound represented by formula (i) and/or formula (ii); 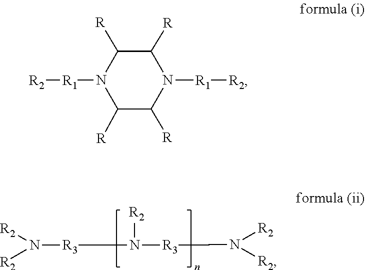
wherein in formula (i), each R is independently selected from H or C1-C3 alkyl; each R₁ is independently selected from 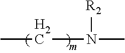
or absence, wherein m is a positive integer selected from 1-3; each R₂ is selected from H or the hydrophobic group, and the molar ratio of the hydrophobic group to N atom in the compound represented by formula (i) is (0.3-0.75):1;
in formula (ii), each R₂ is independently selected from H or the hydrophobic group, the molar ratio of the hydrophobic group to N atom is (0.2-1.5):1, each R₃ is independently selected from substituted or unsubstituted C1-C3 alkylidene; N is a positive integer between 0 and 3.

22. An oil-based drilling fluid, wherein the oil-based drilling fluid comprises a base oil, and a treatment agent comprising the composite emulsifier according to claim 11.

23. The oil-based drilling fluid according to claim 22, wherein the composite emulsifier is contained in an amount of 2-10 parts by weight, based on 100 parts by weight of the base oil.
